(19) United States
(12) Patent Application Publication
(10) Pub. No.: US 2005/0147667 A1
(43) Pub. Date: Jul. 7, 2005

(54) Method of ensuring patient identification of prescribed drug and quantity for prescribed purpose and improved self-identifying drug appearance

(76) Inventor: Robert H. Rines, Boston, MA (US)

Correspondence Address: RINES & RINES, 81 N. STATE STREET, CONCORD, NH, 03301, US

(21) Appl. No.: 10/752,364

(22) Filed: Jan. 6, 2004

(51) Int. Cl. A61K 49/00; A61K 9/20

(52) U.S. Cl. 424/464

(57) ABSTRACT

A technique for assuring patient use of the correct drug pill for a prescribed treatment of a particular body organ or part, involving shaping the pill to have an appearance resembling such body organ—in particular, appropriate prophylactic-dose aspirin pills of heart-shape, as well as other drug pills for treating other body parts the shape of which is imparted to the respective pills.

### FIELD

The present invention is concerned with the problem of ensuring that a patient can readily identify by distinctive appearance the prescribed quantity of a drug that is to be used for a prescribed purpose, being more particularly concerned with the rendering of such identification and described purpose substantially fool-proof.

### BACKGROUND

Throughout the centuries of medicinal drug administration, physicians' offices, hospitals and nursing and pharmaceutical dispensing institutions have instituted varied mechanisms for trying to ensure that the absolutely correct prescribed quantity or dosage of a correct drug formulation is administered to a patient for various prescribed purposes; and, when a drug is entrusted to the care of the patient himself or herself in the home or elsewhere, that the patient can correctly identify the drug, its quantity and required dosages and its intended usage for a prescribed purpose or regimen. Among the principal prior techniques offered and widely used, and to a great degree workable with the exercise of some thought, intelligence, care, and the taking of appropriate time, are the labeling of containers or packages; and sometimes, as in the case of pre-prepared tablets or pills, color coding, or even special shaping of the pill. Unfortunately, this is not fool-proof and it depends upon the attention given at the moment by the drug dispenser and/or the patient.

Though a nurse or a patient may even note the color or other appearance of a pill, moreover, such doesn't always refresh the mind as to the specific prescribed quantity or dosage or even medicinal purpose for which the pill has been prescribed, particularly if multiple different medical drug pills are being used. This phase of the problem has been addressed over the years, and with considerable success, though by no means fool-proof, by pre-filled compartmented pill-dispensing boxes and the like, wherein the compartments are emptied in a planned sequence or ritual; for example, morning, afternoon and/or evening pill-filled compartments, or daily, etc.

Up until the present invention, however, it is believed that there has not been a fool-proof last-minute ensuring identification and automatic “no-brainer” check available that, indeed, the correct medicinal drug and dosage is about to be used and for the correct prescribed medical treatment purpose.

It is to the solution of this particular phase of the problem of drug administration, accordingly, that the present invention is directed.

While more generically applicable, the invention is particularly, though not exclusively, concerned with the many millions of people for whom the medical profession has today prescribed both daily prophylactic and daily treatment of only about 81 milligrams of acetylsalicylic acid (aspirin) for low-dose treatment of the heart. The industry has accordingly relatively recently reacted with special low-dose but conventionally shaped circular substantially flat pills with slightly rounded top and bottom faces (sometimes colored) for this specific purpose—but these are not the customary full-dose aspirin pills of the medicine cabinet to which the world has became accustomed over many decades, and which contain about 325 milligrams (four times the prophylactic purpose dosage) for the very different medical purpose of effectively relieving headaches and pain and fever. The unwitting and erroneous misapplication of these customary strong “headache” aspirin dosages on a daily basis for the very different low-dosage prophylactic heart benefit use, indeed, has provided stomach disorders and other deleterious side effects to significant numbers of sensitive persons. The warnings published by one of the leading aspirin manufacturers, Bayer, include contraindication of use where the user has ulcers or stomach problems such as heartburn, upset stomach, or stomach pain” (“Original Strength” bottle labels of 8/03). Even for their intended pain-relieving purposes, this usual “headache” or “fever” aspirin has sometimes been coated with a so-called “enteric safety” coating and also with “delayed release” to inhibit these stomach difficulties. And, commonly, the erroneous misapplication of the daily low dosage prophylactic 81 gram pill—“adult low strength”—a phrase used both by Bayer and the St. Joseph brand of McNeil-PPG, Inc., Fort Washington, Pa.—for the different purpose of relieving headaches and other pain, falls far short of full efficacy for such purposes.

The present invention provides for the first time, it is believed, the above-mentioned last minute insurance and positive identification that the correct aspirin dosage is about to be swallowed or chewed, and for the correct prescribed medical treatment purpose. As later more fully explained, this result is attained, in accordance with the invention, by physically shaping the pill in the unmistakable shape of the organ or body part it is intended to treat—in this case, the shape of the heart itself. Seeing the heart-shaped pill before swallowing ensures that the patient is taking precisely the correct aspirin dosage for precisely the correct prescribed purpose of heart prophylaxis.

### OBJECTS OF INVENTION

The principal object of the invention, accordingly, is to provide a new and improved method of ensuring patient identification of a prescribed drug quantity, (such as the 81 gram aspirin dosage) for its prescribed purpose (as a daily heart prophylactic), and an improved self-identifying drug appearance that obviates the misapplication and related problems existing for decades, if not centuries, of prior art medicinal drug mis-administration; and, to the contrary, provides a foolproof and even last-minute check ensuring identification that the correct medicinal drug is about to be consumed and for the absolutely correct prescribed medicinal purpose.

A further object is to provide a novel medicinal pill or tablet or the like, the shape of which is made to correspond to the shape of the organ or other body part it has been prescribed for treatment by the pill.

Other and further objects will be explained hereinafter and are more particularly delineated in the appended claims.

### SUMMARY

In summary, however, from its generic viewpoint, the invention embraces a method of ensuring patient identification of a prescribed drug and quantity or dosage for a prescribed purpose of treating a particular body organ or part, that comprises, compacting a prescribed quantity or dosage of a drug material into a solid and fixed permanent pill or tablet and shaping the outer appearance of the pill or tablet to correspond to or resemble the shape or distinctive features of the shape of the particular body organ or part that it is intended to treat with the drug; thereby enabling the patient unambiguously to identify before consuming the pill or tablet that such is the precise prescribed drug pill or tablet and dosage that is precisely intended for treating the prescribed body organ or part.

Preferred and best mode embodiments are hereinafter more fully described in detail.

### DRAWINGS

The invention will now be described with reference to the accompanying drawings,

FIG. 1 of which is an isometric view upon a much enlarged scale of an illustrative application of the invention to the exemplary prophylactic heart aspirin presentation;

FIG. 2 is a similar view of a modification;

FIG. 3 is an isometric of a pill intended for erectile penile blood flow acceleration; and

FIG. 4 is a pill shaped, in accordance with the invention, for use for osteoporosis or similar bone treatment identification.

### DESCRIPTION OF PREFERRED EMBODIMENT(S) OF INVENTION

In accordance with the before-described concept of the invention of physically shaping the pill (or a prominent feature or part thereof) in a shape corresponding to the shape (or prominent feature or part thereof) of the organ or body part it is intended to treat with the pill, the substantially fool-proof and last-minute ensuring identification unambiguously of the prescribed drug (and quantity or dosage) for the prescribed purpose is attained. This concept enables the pill itself to provide fool-proof self-identifying appearance.

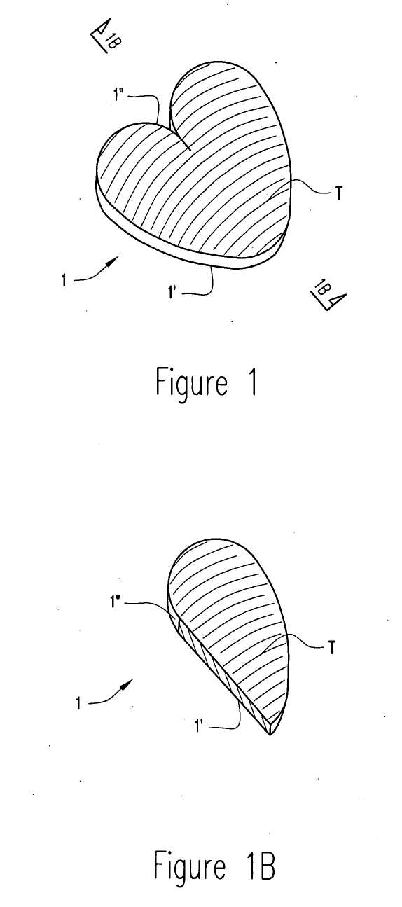

For the important case of low-strength adult aspirin dosage for heart prophylactic treatment, the pill construction and appearance of FIG. 1 may be provided wherein the normally circular cylindrical pill body 1, with its before-mentioned normal flat or slightly convex curved top and bottom surfaces (such as the bottom 1¹) is modified by providing an open wedge 1¹¹ to assume the self-identifying shape or appearance resembling the outline shape of the upper part of the heart as the intended organ for prophylactic treatment by this pill.

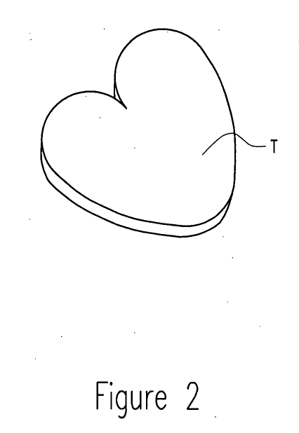

To minimize production change-over costs in the conventional pill-forming machinery, and to enable the precise required pre-designated 81 milligram dose to be provided by the aspirin-composited compressed and compacted solid permanent pill, despite the missing volume of the open wedge—and without changing the size of the pill (and the machinery cavities)—the pill may be formed with, for example, a slight top surface dome or thickening T to provide the overall required grains of aspirin to assure the precise 81 milligram dosage prescribed. Other regions of the pill for packing the grains eliminated by the wedge 1¹¹ or other shaping that imparts the appearance of the heart organ intended to be treated, may also be used, as desired. In fact, the pill may be shaped elsewhere, as shown in the modification of FIG. 2, again using a thicker dome T to complete the 81 milligram volume dosage presently provided in the conventional cylindrical pill.

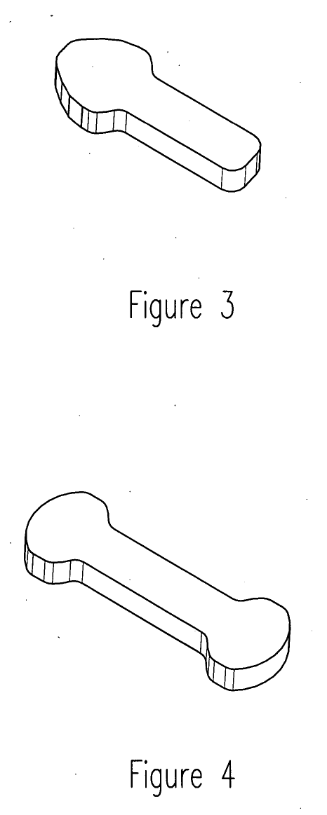

In FIG. 3, as another illustration, the tablet is shown somewhat shaped to resemble the outline of a penis unambiguously to ensure identification, even at the last minute before consumption, that this is the correct pill for the intended prescribed dosage of an erectile blood flow acceleration drug such as marketed under the trademark Viagra, and others. The normal “Viagra”, “Pinagra”, etc. pills are 100 milligram dose oval tablets of compacted solid sildenafil citrate, which, in accordance with the invention, would be modified in shape, as for example in FIG. 3, distinctively to have penile outline shape features or characteristics and thereby provide the visual assurance that the right pill is being consumed for the correct intended organ treatment.

As still another example, a prescribed dose of “bisphosphonates (alendronate and risedronate)“or other similar drug may be provided in the pill or tablet that is somewhat bone-shaped as in FIG. 4, to ensure positive identification before being swallowed for the prescribed and intended treatment of osteoporosis of the bones or the like.

This invention is not limited to generally cylindrical pills but may be used in more spherical and more elongated forms, as well; all, however, in accordance with the present invention, shaped to incorporate contours simulating appearance features of the body pert to be treated.

Further modifications will also occur to those skilled in this art, such being considered to fall within the spirit and scope of the invention as defined in the appended claims.

1. A method of ensuring patient identification of a prescribed drug and quantity or dosage for a prescribed purpose of treating a particular body organ or part, that comprises, compacting a prescribed quantity or dosage of a drug material into a solid and fixed permanent pill or tablet and shaping the outer appearance of the pill or tablet to correspond to or resemble the shape or distinctive features of the shape of the particular body organ or part that it is intended to treat with the drug; thereby enabling the patient unambiguously to identify before consuming the pill or tablet that such is the precise prescribed drug pill or tablet and dosage that is precisely intended for treating the prescribed body organ or part.

2. The method of claim 1 wherein the drug is aspirin, and the body organ is the heart, the prescribed quantity or dosage is 81 milligrams prescribed for heart prophylactic treatment, and wherein the pill or tablet is shaped to resemble the outline shape of the heart, thereby unambiguously to enable the patient to distinguish from higher dosage aspirin pills intended for treating other body ailments, or other pills.

3. An 81 milligram fixed dosage compacted generally cylindrical pill shaped in substantially heart-shaped outline and designed for heart prophylactic treatment.

4. A fixed dosage compacted generally cylindrical pill shaped in substantially penile shaped outline, and comprised of an erectile blood-flow acceleration drug material.

5. A fixed dosage compacted generally cylindrical pill shaped in substantially the outline shape of a bone, and comprising a drug material prescribed for treating osteoporosis.
